(19) United States
(12) Patent Application Publication
(10) Pub. No.: US 2016/0022376 A1
(43) Pub. Date: Jan. 28, 2016

(54) MIRROR, IN PARTICULAR MEDICAL MIRROR, AND DEVICE, IN PARTICULAR FOR ROOT CANAL TREATMENT

(71) Applicant: Wolfgang WINKELMANN, Tübingen (DE)

(72) Inventor: Wolfgang WINKELMANN, Tübingen (DE)

(21) Appl. No.: 14/772,093

(22) PCT Filed: Feb. 4, 2014
(86) PCT No.: PCT/EP2014/052094

§ 371 Date: Sep. 2, 2015

(30) Foreign Application Priority Data
Mar. 7, 2013 (DE) 20 2013 002 440.2
Mar. 15, 2013 (DE) 10 2013 004 876.1

(51) Int. Cl. A61C 1/08; A61B 17/17; A61C 5/02; A61C 5/04; A61B 1/247; A61C 1/12

(52) U.S. Cl. CPC A61C 1/082; A61B 1/247; A61C 1/12; A61C 5/025; A61C 5/04; A61B 17/1703; A61B 2019/5221

(57) ABSTRACT

The present invention relates to novel mirrors, in particular medical mirrors, preferably dental mirrors, also referred to as mouth mirrors, which have a hole through which dental instruments, in particular root canal instruments or gutta-percha points, can be placed, and to devices comprising a dental mirror according to the invention.

The present invention relates to novel mirrors, in particular medical mirrors, preferably dental mirrors, also referred to as mouth mirrors, which have a hole through which dental instruments, in particular root canal instruments or gutta-percha points, can be placed, and to devices comprising a dental mirror according to the invention.

Mirrors are used frequently during surgeries, in particular also during endodontic surgeries, in order to easily see the regions of the body to be treated, such as teeth, and the operating steps. While an operating step is carried out, the appropriate site should be visible at the same time to the treating physician or dentist via the mirror. Corresponding medical mirrors are known.

Dental mirrors are also sufficiently known. They typically have a round mirror surface, which may be planar or concave. The mirror surface is located on a mirror base body, on which usually a handle piece is provided. Typical diameters of the mirror surfaces of dental mirrors are 21 mm (size 4) through 24 mm (size 5) to 30 mm (size 8).

During surgeries in regions of the body that are difficult to see, for example the molars, it is almost impossible to use the instruments necessary for the surgery and, at the same time, a medical mirror, in particular a dental mirror, at the surgical site, wherein the mirror at the same time is intended to render the corresponding site visible to the treating physician.

The technical problem underlying the present invention is that of providing a medical mirror, in particular a dental mirror, which can be used during surgeries and treatments in difficult-to-access body regions without impairing the use of surgical instruments, yet at the same time renders the operating step easily visible to the treating physician.

The present invention solves the underlying technical problem by providing a mirror according to claim 1 and a device, comprising a mirror according to the invention, according to claim 15.

The present invention solves the underlying technical problem in particular by providing a mirror, comprising a mirror surface, wherein the mirror surface has a hole. The hole thus extends through the mirror base body, which carries the mirror surface.

The mirror is preferably a medical mirror. The mirror is particularly preferably a dental mirror, also referred to as a mouth mirror.

The mirror surface is preferably borne by a mirror base body in the known manner, which is to say the mirror base body is preferably coated at least on one side with a mirror surface.

The hole can advantageously be used to allow a surgical instrument, in particular an elongated surgical instrument, such as often used dental instruments or a gutta-percha point, to be pushed through the hole and thus extend through the hole, in particular during the treatment. The mirror thus surrounds the surgical instrument and reflects the surgical site, so that the physician is able to easily see the same. The surgical instrument can be used without restrictions in the process, and the mirror can be tilted along the axis of the surgical instrument in such a way that the physician is provided with a substantially good picture.

In one preferred embodiment, the hole has a diameter of at least 1 mm and of no more than 11 mm. In one preferred embodiment, the hole has a diameter of at least 1 mm. In one preferred embodiment, the hole has a diameter of at least 2 mm. In one preferred embodiment, the hole has a diameter of no more than 7 mm. In one preferred embodiment, the hole has a diameter of no more than 5 mm. In one preferred embodiment, the hole has a diameter of at least 1 mm and of no more than 3 mm.

The diameter of the hole must be at least as large as the diameter of the surgical instrument which is to extend through the hole. The diameter of the hole is preferably slightly larger than the diameter of the surgical instrument which is to extend through the hole, so that the mirror may thus be tilted along the axis of the surgical instrument so as to be able to adjust the projected image and provide particularly good visibility to the physician of the site to be treated via the reflected image.

In one preferred embodiment, the mirror surface has a maximum diameter of no more than 5 cm. In one preferred embodiment, the mirror surface has a maximum diameter of no more than 3.5 cm. In one preferred embodiment, the mirror surface has a maximum diameter of no more than 2.5 cm. In one preferred embodiment, the mirror surface has a diameter of at least 0.5 cm.

In one preferred embodiment, the hole is located approximately at the center of the mirror surface. In one preferred embodiment, the hole is centered at the center of the mirror surface.

In one preferred embodiment, the hole has flattened or rounded edges on the back of the mirror base body bearing the mirror surface. This advantageously improves the tilting ability of the mirror along the axis of the surgical instrument extending through.

In one preferred embodiment, the mirror surface is round. In one preferred embodiment, the basic shape of the mirror surface is round. It goes without saying, however, that the mirror surface can take on any other shape, for example the mirror surface can be oval, triangular, square, quadrangular or polygonal.

In one preferred embodiment, the mirror surface has at least one concave recess or notch. In one preferred embodiment, the mirror surface has a concave recess. In one alternative embodiment, the mirror surface has two concave recesses.

The first recess or notch can advantageously be used to allow in particular a dental mirror to be moved as close as possible to the work area, for example to the root canal entrances of a tooth. The second recess or notch can advantageously be used so that the surgical instrument extending through the hole, for example the drill head of a contra-angle handpiece, has the largest possible degree of freedom, in particular when the mirror is bent along the instrument axis.

In one preferred embodiment, the mirror, in particular the dental mirror, comprises a handle piece. As an alternative, it may also be provided that no handle piece is present, for example when the mirror is used in an alternative embodiment of the device according to the invention, in which the mirror is attached to a surgical instrument, in particular a contra-angle handpiece.

In one preferred embodiment, the center of the at least one concave recess is located at an angle of 0° to 90°, in particular of approximately 45°, with respect to the longitudinal axis of the handle piece.

In one preferred embodiment, the hole is connected to the mirror surface outer edge via a gap or a slot. This allows easy insertion or removal of a surgical instrument. The width of the gap or of the slot is preferably smaller than the diameter of the hole, but larger than the diameter of the surgical instrument.

In one preferred embodiment, the gap opens into a concave recess of the mirror surface.

In one preferred embodiment, the mirror surface is a planar mirror surface. In one alternative embodiment, the mirror surface is concave. In one alternative embodiment, the mirror surface is a magnifying mirror surface.

In one preferred embodiment, the dental mirror comprises an insulated electrical connection at the handle piece which is connected via an insulated electrical cable to a contact area produced from an electrically conducting material, wherein the contact area is located on the inner wall surface of the hole or at the back of the mirror surface. In this way, an electronic measurement of the root canal depth is advantageously possible, without having to move an electrode close to the root canal instrument in an undesirable manner.

The present invention also solves the underlying technical problem by a device for treating teeth, comprising a mirror according to the invention, in particular a dental mirror according to the invention, a contra-angle handpiece, and a dental instrument connected to the contra-angle handpiece, wherein the dental instrument extends through the hole of the dental mirror.

In one preferred embodiment, the device is used for the endodontic treatment of teeth and comprises a dental mirror according to the invention, a contra-angle handpiece, and a root canal instrument connected to the contra-angle handpiece, wherein the root canal instrument extends through the hole of the dental mirror.

In one preferred embodiment, the root canal instrument is a nerve broach, a file, a cutter, a drill or a reamer.

In one preferred embodiment, the mirror is assigned to the dental instrument, in particular to the root canal instrument, and to the contra-angle handpiece. In one alternative embodiment, the mirror is connected to the contra-angle handpiece.

In one preferred embodiment, the dental instrument, in particular the root canal instrument, makes contact with the inner surface of the hole and/or the back of the mirror surface. In particular in the embodiment of the dental mirror in which the dental mirror comprises an insulated electrical connection at the handle piece which is connected via an insulated electrical cable to a contact area produced from an electrically conducting material, wherein the contact area is located on the inner wall surface of the hole or at the back of the mirror surface, in this way electronic measurement of the root canal depth is possible.

The present invention also solves the underlying technical problem by a mirror according to the invention for use during a medical operation. The mirror is preferably used in such a way that a surgical instrument extends through the hole of the mirror.

The present invention also solves the underlying technical problem by a dental mirror according to the invention for use for the treatment of teeth. The dental mirror is preferably used in such a way that a dental instrument extends through the hole of the dental mirror.

The present invention also solves the underlying technical problem by the use of a mirror according to the invention during a medical operation. The mirror is preferably used in such a way that a surgical instrument extends through the hole of the mirror.

The present invention also solves the underlying technical problem by the use of a dental mirror according to the invention for the treatment of teeth. The dental mirror is preferably used in such a way that a dental instrument extends through the hole of the dental mirror.

The present invention also solves the underlying technical problem by methods for medical treatment, comprising the steps of a) passing a surgical instrument through the hole of the mirror according to the invention, and b) using the surgical instrument at a site to be treated and simultaneously observing the site to be treated via the mirror.

The present invention also solves the underlying technical problem by methods for dental treatment, comprising the steps of a) passing the dental instrument or a gutta-percha point through the hole of the dental mirror according to the invention, and b) using the dental instrument at a tooth to be treated and simultaneously observing the tooth to be treated via the dental mirror.

The present invention is described in more detail hereafter by way of example based on the following figures, without the figures being interpreted to be limiting.

In the drawings:

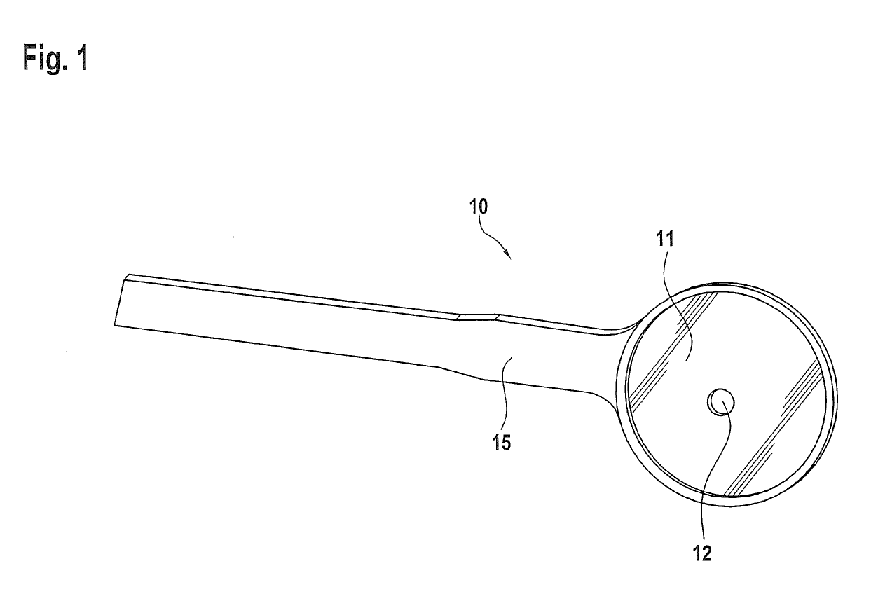

FIG. 1 shows a dental mirror according to the invention comprising a handle piece;

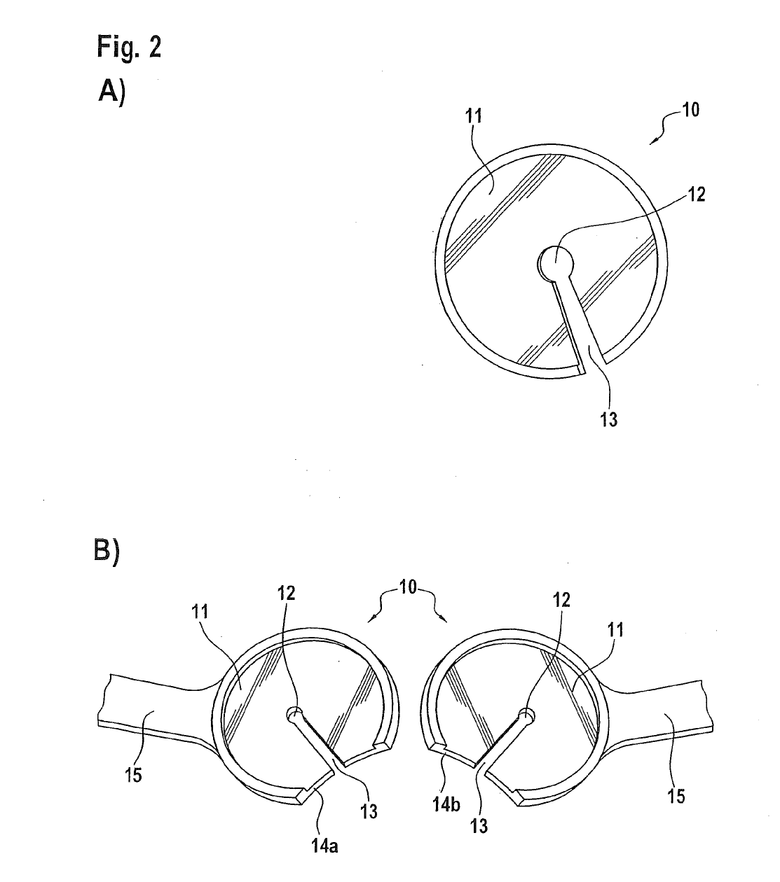

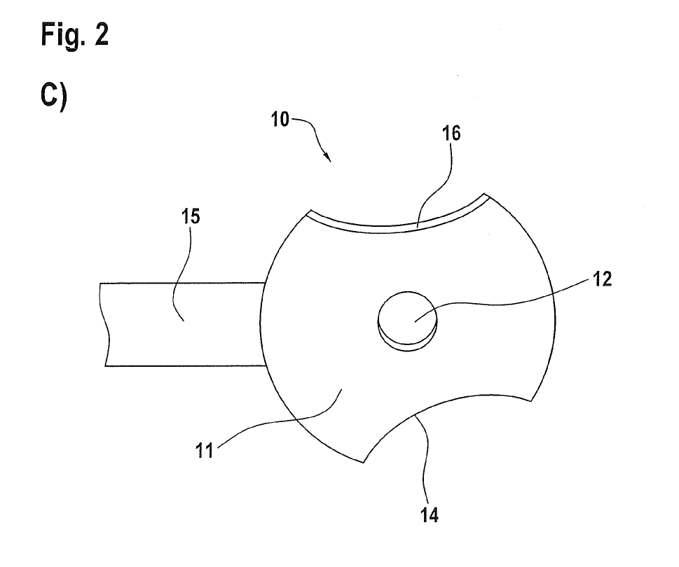

FIG. 2A shows a dental mirror according to the invention having a gap;

FIG. 2B shows a dental mirror according to the invention having a gap, a recess, and a handle piece;

FIG. 2C shows a dental mirror according to the invention having two concave recesses and one handle piece;

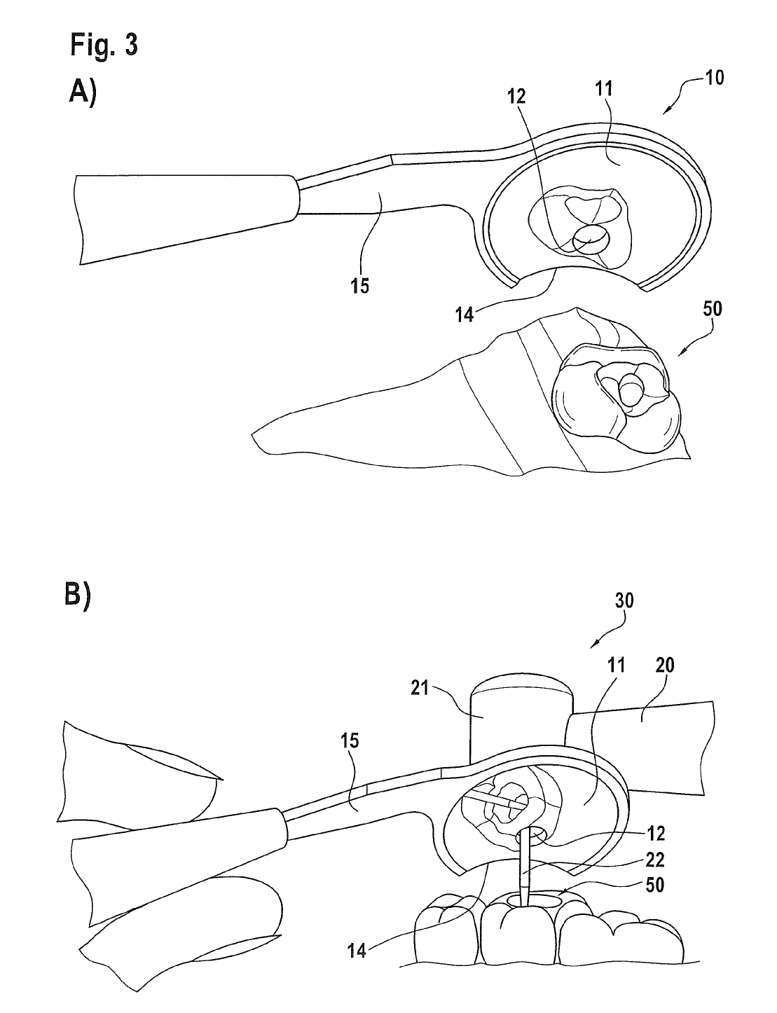

FIG. 3A shows a dental mirror according to the invention having a concave recess and a handle piece prior to the treatment of a tooth;

FIG. 3B shows a dental mirror according to the invention having a concave recess and a handle piece during the treatment of a tooth using a dental instrument;

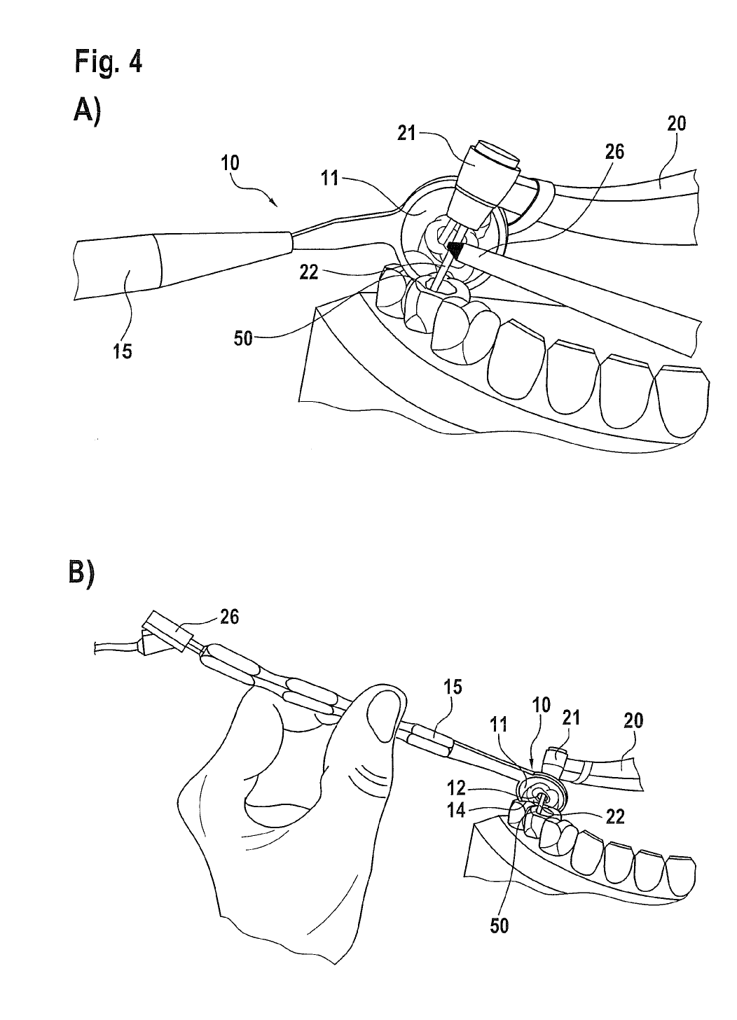

FIG. 4A shows a conventional treatment of a tooth using a dental mirror from the prior art and conventional electronic root canal depth measurement;

FIG. 4B shows a treatment according to the invention and electronic root canal depth measurement of a tooth using a dental mirror according to the invention; and

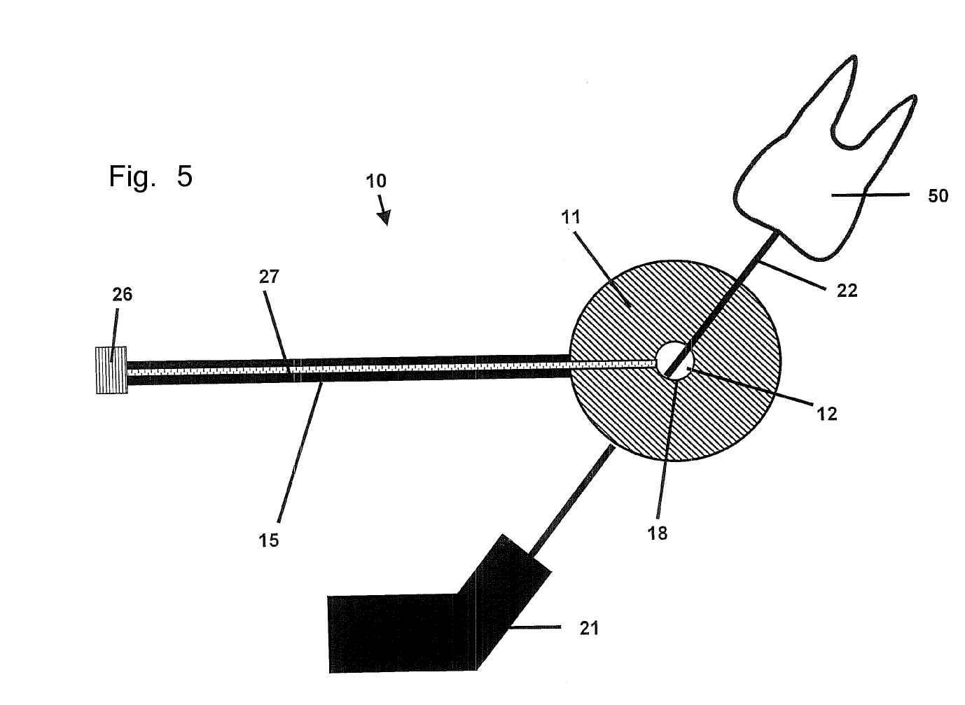

FIG. 5 shows a device according to the invention comprising a mirror according to the invention and a root canal instrument extending through the mirror, the instrument being connected to a contra-angle handpiece.

FIG. 1 shows a dental mirror (10) according to the invention. The mirror comprises a mirror surface (11), which is known from the prior art, on a mirror base body and a handle piece (15), which is known from the prior art. The mirror surface of a dental mirror is typically round. The mirror according to the invention is characterized in that the mirror surface (11) has a hole (12), which is preferably positioned approximately centrally in the mirror surface. For example during a root canal treatment, in particular in the case of molars, and most particularly in the case of upper molars, this hole can advantageously be used to pass through an instrument required for the root canal treatment, for example a needle, a cutter, a file or a gutta-percha point, so that the instrument can be used for the treatment and, at the same time, the mirror can be positioned so that the treatment site is easily visible to the dentist. The size of the hole (12) is therefore preferably slightly larger than instruments used for root canal treatment, so that the same fit through the hole and the mirror can be rotated with respect to the axis of the instruments so that the treatment site can be reflected well.

FIG. 2A shows a mirror according to the invention, which again has a hole (12) in the mirror surface (11), wherein a gap (13) extends between the outer edge of the mirror surface (11) and the hole (12). This gap can advantageously be used to laterally insert a dental instrument, for example an instrument used for root treatment, which is usually elongated, into the hole, without having to push the instrument into the hole starting from the tip. It is thus also possible, for example, to remove the mirror during the treatment, without having to pull the instrument out of the treatment site.

FIG. 2B shows two dental mirrors (10) according to the invention, each comprising a mirror surface (11), a handle piece (15), and a hole (12). The hole (12) is again provided with a gap (13) for inserting and removing a dental instrument, wherein the gap (13) opens into a concave recess (14a, 14b). In FIGS. A and B, the gaps (13) and the concave recesses (14a, 14b) are provided laterally reversed. In this way, the appropriate instrument may be selected in each case, depending on whether the treating physician or the assistant holding the mirror holds the same in the right hand or the left hand, and depending on whether a tooth in the right jaw region or in the left jaw region, or in the upper jaw or lower jaw, is treated. The angle at which the gap (13) and/or the center of the recess (14a, 14b) is offset from the longitudinal axis of the handle piece (15) may range from 0° to 90°, wherein an angle around approximately 45°, as shown, is preferred.

FIG. 2C shows a mirror according to the invention, which again has a mirror surface (11) and a handle piece (15), wherein a hole (12) is again integrated into the mirror surface. The mirror (10) has a concave indentation (14). This indentation can advantageously be used to better move the mirror close to the tooth, since the convexity normally resulting from the circular design of a dental mirror is eliminated. In addition, a second convexity (16) is shown, which makes it possible, for example, for a drill head of a contra-angle handpiece to have the largest possible degree of freedom. FIG. 2B shows a mirror according to the invention, which again has a hole (12) in the mirror surface (11), wherein a gap (13) extends between the outer edge of the mirror surface (11) and the hole (12). This gap can advantageously be used to laterally insert a dental instrument, for example an instrument used for root treatment, which is usually elongated, into the hole, without having to push the instrument into the hole starting from the tip. It is thus also possible, for example, to remove the mirror during the treatment, without having to pull the instrument out of the treatment site.

FIG. 3A shows a dental mirror (10) according to the invention having a concave recess (14) and a handle piece (15) prior to the treatment of a tooth (50). The mirror surface (11) reflects the site of a tooth (50) to be treated, wherein the hole (12) due the size and position thereof does not impair the visible range. The concave recess (14) advantageously allows the mirror (10) to be moved close to the tooth (50).

FIG. 3B shows a device (30) according to the invention, comprising a dental mirror (10) according to the invention and a dental instrument (20, 21, 22) during the treatment of a tooth (50). The dental mirror (10) again has the concave recess (14) and the handle piece (15). The dental instrument (30) comprises a root canal instrument (22), for example a nerve broach, a file, a cutter, a drill or a reamer, and a contra-angle handpiece (21) and a root canal instrument (22). During the treatment, the root canal instrument (22) extends through the hole (12) so that the dental mirror (10) is not in the way. However, at the same time, the dentist is able to exactly observe and visually check the treatment progress via the mirror surface (11).

FIG. 4A shows a conventional treatment of a tooth (50) using a dental mirror (10) from the prior art and conventional electronic root canal depth measurement (26). The dental instrument (20) comprises a root canal instrument (22) and a contra-angle handpiece (21). The electrode for root canal depth measurement (26) must be pressed against the root canal instrument (22). The electrode (26) and the mirror (10) are in the way during the treatment under these space constraints, and the dentist can see the site on the tooth (50) to be treated only with difficulty and imprecisely via the conventional mirror.

FIG. 4B shows a treatment according to the invention and electronic root canal depth measurement of a tooth (5) using a dental mirror (10) according to the invention having a mirror surface (11) and a hole (12), and a concave recess (14) and handle piece (15). A dental instrument (20) is also apparent again, comprising an elongated root canal instrument (22), for example a nerve broach, a file, a cutter, a drill or a reamer, and a contra-angle handpiece (21) holding the root canal instrument (22). The root canal instrument (22) extends through the hole (12) of the dental mirror (10) and is used for the endodontic treatment of a tooth (50).

The dental mirror (10) moreover has an insulated electrical connection (26) at the handle piece (15), which via an insulated electrical cable in the interior of the handle piece (15) can be connected the hole (12) to an electronic device for depth measurement. The wall of the hole (12), or a portion of the back of the mirror surface (11) can be made of an electrically conducting material, which is connected to the insulated electrical cable. The root canal instrument (22) can now advantageously make contact with this electrical contact area at the hole (12), so that electronic depth measurement is possible during the treatment, without having to hold the electrode against the root canal instrument for root canal depth measurement. Due to the size of the hole (12), the mirror (10) can be easily rotated, without this adversely affecting the freedom of motion of the root canal instrument (22). The dentist can thus rotate the mirror into a desired position so as to easily see the site at the tooth (50) to be treated in the reflected image. The mirror (10) does not impair the treatment using the root canal instrument (22), and the dental instrument (20) holding the root canal instrument (22) does not impair the view of the tooth (50) via the mirror (10) In addition, no interfering electrode is needed at the treatment site for root canal depth measurement. As a result of the concave recess (14), the mirror (10) can be moved close to the tooth (50) particularly well.

FIG. 5 schematically shows a device according to the invention, comprising a dental mirror (10) according to the invention, an elongated root canal instrument (22), for example a nerve broach, a file, a cutter, a drill or a reamer, and a contra-angle handpiece (21) holding the root canal instrument (22). The dental mirror (10) again comprises a handle piece (15) and a mirror surface (11). The root canal instrument (22) extends through the hole (12) of the dental mirror (10) and is used for the endodontic treatment of a tooth (50). The dental mirror (10) can, of course, have further features described in the present invention, for example the gaps and recesses shown in FIGS. 2 and 3.

The dental mirror (10) moreover has an insulated electrical connection (26) at the handle piece (15), which via an insulated electrical cable (27) can be connected the hole (12) to an electronic device for depth measurement. The wall (18) of the hole (12), or a portion of the back of the mirror surface (11), can be made of an electrically conducting material, which is connected to the cable (27). The root canal instrument (22) can now advantageously make contact with this electrical contact area at the hole (12), so that electronic depth measurement is possible during the treatment, without the root canal instrument being connected to an undesirable electrical connection, for example a cable, or the same having to be held close to the root canal instrument (21). This embodiment of the mirror (10) of the device can, of course, also be provided in a single mirror according to the invention. It may also be provided that the mirror does not comprise a handle piece, but is adjustably and rotatably connected to the contra-angle handpiece.

1-19. (canceled)

20. A medical mirror, comprising:
a mirror surface,
wherein the mirror surface has a hole.

21. The medical mirror according to claim 20, wherein the mirror is a dental mirror.

22. The medical mirror according to any claim 20, wherein the hole has a diameter of at least 1 mm and of no more than 11 mm.

23. The medical mirror according to claim 20, wherein the mirror surface has a maximum diameter of no more than 3.5 cm.

24. The medical mirror according to claim 20, wherein the hole is located approximately at a center of the mirror surface.

25. The medical mirror according to claim 20, wherein the hole has flattened or rounded edges at the back of the mirror surface.

26. The medical mirror according to claim 20, wherein the mirror surface is round.

27. The medical mirror according to claim 20, wherein the mirror surface has at least one concave recess.

28. The medical mirror according to claim 21, wherein the dental mirror further includes a handle piece.

29. The medical mirror according to claim 27, wherein the center of the at least one concave recess is located at an angle of 0° to 90°, with respect to the longitudinal axis of the handle piece.

30. The medical mirror according to claim 27, wherein the center of the at least one concave recess is located at an angle of approximately 45°, with respect to the longitudinal axis of the handle piece.

31. The medical mirror according to claim 20, wherein the hole is connected to the mirror surface outer edge via a gap.

32. The medical mirror according to claim 31, wherein the gap opens into a concave recess.

33. The medical mirror according to claim 20, wherein the mirror surface is a magnifying mirror surface.

34. The medical mirror according to claim 28, wherein the dental mirror comprises an insulated electrical connection at the handle piece which is connected via an insulated electrical cable to a contact area produced from an electrically conducting material, the contact area being located on the inner wall surface of the hole or at the back of the mirror surface.

35. A device for treating teeth, comprising:
a medical mirror including a mirror surface having a hole;
a contra-angle handpiece, and
a dental instrument connected to the contra-angle handpiece,
wherein the dental instrument extends through the hole of the dental mirror.

36. A device for the endodontic treatment of teeth, comprising:
a medical mirror including a mirror surface having a hole;
a contra-angle handpiece; and
a root canal instrument connected to the contra-angle handpiece,
wherein the root canal instrument extends through the hole of the dental mirror.

37. The device according to claim 35, wherein the root canal instrument is selected from a group consisting of:
a nerve broach, a file, a cutter, a drill or a reamer.

38. The device according to claim 36, wherein the mirror is connected to the contra-angle handpiece.

39. The device according to claim 36, wherein the root canal instrument comes in contact with an inner surface of the hole and/or the back of the mirror surface.
